(19) United States
(12) Patent Application Publication
(10) Pub. No.: US 2005/0170512 A1
(43) Pub. Date: Aug. 4, 2005

(54) Method for cleaning reaction vessels in place

(76) Inventors: Sean Rubin, Boston, MA (US); Sarah Hembree, Boston, MA (US); Dina Haines, Hudson, MA (US); Steven J. Gordon, Weston, MA (US)

Correspondence Address: PERMAN & GREEN, 425 POST ROAD, FAIRFIELD, CT, 06824, US

(21) Appl. No.: 10/975,824

(22) Filed: Oct. 28, 2004

Related U.S. Application Data
(60) Provisional application No. 60/515,235, filed on Oct. 28, 2003.

(51) Int. Cl. G01N 35/00

(52) U.S. Cl. 436/43; 422/63

(57) ABSTRACT

A method and system for reusing reaction vessels in a biotechnical processing apparatus. The system has a frame, an array of reaction vessels, an automatic actuator, and a decontamination system. The frame has at least one processing station. The array of reaction vessels is mounted to the frame for effecting biotechnical processing at the precessing station. The automatic actuator is connected to the frame and the reaction vessels for dispersion of solution in and out of the reaction vessels. The decontamination system is constructed in the frame for being accessed by the actuator to decontaminate the reaction vessels. The decontamination system includes a wash section, a heating section, a supply of wash fluid, a supply of acid solution and a supply of base solution. The actuator is programmed to access the decontamination section to subject the access the decontamination section, a bas solution and a wash reagent for decontamination the reaction vessels.

### RELATED APPLICATIONS

This application claims priority from U.S. Provisional Patent Application No. 60/515,235 filed Oct. 28, 2003 incorporated by reference herein in its entirety.

### BACKGROUND OF THE INVENTION

1. Field of the Invention

This disclosure relates to procedures for the in situ cleaning of material handling and reaction vessels so that they may be quickly and efficiently re-used for applications that usually require single use disposable vessels.

2. Brief Description of Related Developments

The reuse of reaction vessels can result in a significant cost saving for applications involving multiple samples and/or sequential processes.

Current conventional methods for cleaning reusable vessels involve a cleaning or decontamination protocol that is not convenient or is too lengthy to implement directly on the instrument performing the material transfer or reaction. These methods require removal of the vessels from the automated equipment, with concomitant time and labor expenditures.

Many of the most stringent requirements for clean, uncontaminated vessels are present to biotechnological applications where very small quantities of reagents are handled and where many operations require the amplification of very small quantities of the starting materials. In such applications, contaminants present in the vessels are amplified along with the starting materials during the processing of the samples. When amplified, the small quantity of contaminant, contributes a high background signal which may at times block out the signal derived from the sample under test.

Procedures for the handling of liquids containing samples that are intended to undergo enzymatic amplification reactions have the most stringent requirements for clean vessels. For example, an enzymatic reaction that replicates a molecule many times can generate a significant number of molecules from a single molecule. One commonly used enzymatic amplification process is the polymerase chain reaction [PCR]. This reaction has proven to be particularly sensitive to contamination in a reusable vessel, to the extent that it is commonly performed in single use equipment.

PCR is a technique used in genomic research and diagnostics for amplifying particular DNA fragments at a geometric rate. PCR can be utilized in highly automated processes with robotic or semi-robotic systems handling the manipulation of multiple samples through the processing steps. Although the vast majority of researchers use disposable pipettes and disposable reaction vessels to avoid any contamination from one reaction to the next, a variety of techniques have been reported in the literature for cleaning reusable vessels.

Some other carryover elimination methods have been developed, which alter some of the reagents used in the PCR reaction causing undesired results. However, the most conventional way for preparing laboratory ware for re-use in PCR applications continues to be a cleaning protocol, where the contaminated vessels are removed from the automated sample processing system and rigorously cleaned offline before being replaced in the automated system.

It would be desirable to provide an automated materials handling system for processing very small volume samples with the means to decontaminate the used reaction vessels so that they can be reused. In particular it would be desirable to provided a method of minimizing the downtime for cleaning of automated equipment handling minute quantities of biological materials, especially where those biological materials are subjected to amplification steps, such as enzymatic amplification steps.

### BRIEF SUMMARY OF THE EXEMPLARY EMBODIMENTS

In accordance with a first exemplary embodiment a system for reusing reaction vessels in a biotechnical processing apparatus is provided. The system has a frame, an array of reaction vessels, an automatic actuator, and a decontamination system the frame has at least one processing station. The array of reaction vessels is mounted to the frame for effecting biotechnical processing at the processing station. The automatic actuator is connected to the frame and the reaction vessels for dispersion of solution in and out of the reaction vessels. The decontamination system is constructed in the frame for being accessed by the actuator to decontaminate the reaction vessels. The decontamination system includes a wash section, a heating section, a supply of wash fluid, a supply of acid solution and a supply of base solution. The actuator is programmed to access the decontamination section to subject the reaction vessels to an acid solution, a base solution and a wash reagent for decontaminating the reaction vessels.

In accordance with another exemplary embodiment, a method is described for decontaminating reaction vessels used in a biotechnical application in which, during a biotechnical process, multiple reaction vessels are moved by robot through multiple biotechnical processing stations positioned on a workdeck, such as a polymerase chain reaction. The used reaction vessels are moved to a wash station, located on the workdeck, where they are flushed with a reagent wash.

The robot moves the reaction vessels to an acid wash station to aspirate an acid solution into the reaction vessels. Thereafter, the robot moves the acid filled reaction vessels to a thermal station, where the acid solution is heated in the reaction vessels. The robot moves the heated reaction vessels to a wash station, where the acid solution is dispensed from the reaction vessels.

The robot moves the reaction vessels to a base wash station and a base solution is aspirated into the reaction vessels. The reaction vessels are then moved back to the wash station to enable the reaction vessels to be flushed with a wash reagent.

Apparatus is also described comprising a system for performing biotechnical processing that includes a work deck on which multiple processing stations are constructed. A robot, on which is mounted an array of reaction vessels is movable to enable the presentation of the reaction vessels to the processing stations.

The processing stations include decontamination stations constructed in the work deck in a position accessible to the robot. The decontamination stations include: a wash station connected to receive and remove fluids; a thermal station having a source of heat; a supply of wash reagent, a supply of acid solution and a supply of base solution connected for application to the reaction vessels.

The robot is programmed to move said array of reaction vessels over said work station to sequentially subject the reaction vessels to: a wash reagent, an acid solution, hear, a base solution, and a wash reagent to decontaminate the reaction vessels.

### BRIEF DESCRIPTION OF THE DRAWING

The invention is described in more detail below with reference to the attached drawing in which:

FIG. 1 is a perspective view of the automated system incorporating features of an exemplary embodiment of the invention;

FIG. 2 is a perspective view of the components of the workdeck in the system shown in FIG. 1;

FIG. 3 is a perspective view of a portion of a robot, holding an array of capillary reaction vessels, used in the system of FIG. 1;

FIG. 4 is a perspective view of the wash station of the system shown in FIG. 1;

FIG. 5 is a perspective view of the thermal cycle station of the system shown in FIG. 1;

FIG. 6 is a block diagram of the components of the system of shown in FIG. 1;

FIG. 7 is a flow diagram of the process carried out by the system of FIG. 1;

FIG. 8 is a block diagram of the components of the system in accordance with another exemplary embodiment of the system of this invention; and

FIG. 9 is a schematic diagram of an automated system in accordance with yet another exemplary embodiment.

### DETAILED DESCRIPTION OF THE EXEMPLARY EMBODIMENTS

A system is provided for performing automated biotechnological processes, such as a polymerase chain reaction (PCR). Such processes use reaction vessels, such as capillaries/pipettes/tubes. As part of this system an apparatus and process is provided to decontaminate the reaction vessels after each use without removing the vessels from the system. The cleaning process is performed as part of the automated operation of the system.

The decontamination process for cleaning a vessel entails a partial or complete filling of the vessel with an acid, heating the acid and the vessel, agitating the fluid to bath the vessel surfaces with the acid, elimination of the acid, partial or complete filling of the vessel with a base, agitation, if required, to bath the vessel surfaces with the base, elimination of the base, and rinsing the vessel with a wash reagent or water. The process is accomplished automatically.

The vessel may be glass or an material non-reactive to the cleaning reagents.

The acid may be any acid, especially a strong acid. Acids such as nitric, sulfuric or hydrocholic are preferred with nitric acid being most preferred. The concentration of nitric acid solution may be in the range from about 0.001 to about 15 molar. A molarity in the range of from about 1 and 15 is preferred with a molarity from about 5 and 10 being most preferred.

The base may be especially a strong base. Acids such as alkali or alkaline earth hydroxides are suitable with alkali metal hydroxides being preferred and sodium or potassium hydroxide being most preferred. The normality of the base may range from about 0.001 to about 5; preferably from about 0.1 to about 5; most preferably from about 2 to about 5.

The heating step is performed at a temperature in the range of from about 28 to about 200° C.; preferably in the range of from about 70 to about 200° C.; and most preferred in the range of from about 100° to about 200° C.

It will be appreciated by those skilled in the art that the optimum and maximum temperature will vary with the type of material of the vessel being cleaned; that the degree of cleaning is a function of both time and temperature; and that cleaning will occur more quickly at higher temperatures.

In one exemplary embodiment, an automated workstation for assembling and performing PCR operations is adapted to perform a process for cleaning PCR reaction vessels. In the exemplary system, the reaction vessel is a glass capillary tube that is sealed on one end with a Teflon plunger and at the other with an elastomer seal pad. The glass capillaries are housed within a nano-pipetter. After a PCR reaction is completed, the above steps are performed. Although the cleaning protocol is described with glass capillaries, it should be understood that this invention might be applied to vessels that are compatible with the cleaning reagents.

Although this application, PCR, is being described as an example, it should be understood that the present invention may be used for any number of enzymatic reactions or non-enzymatic reactions where vessels need to be decontaminated whether amplification is occurring or not.

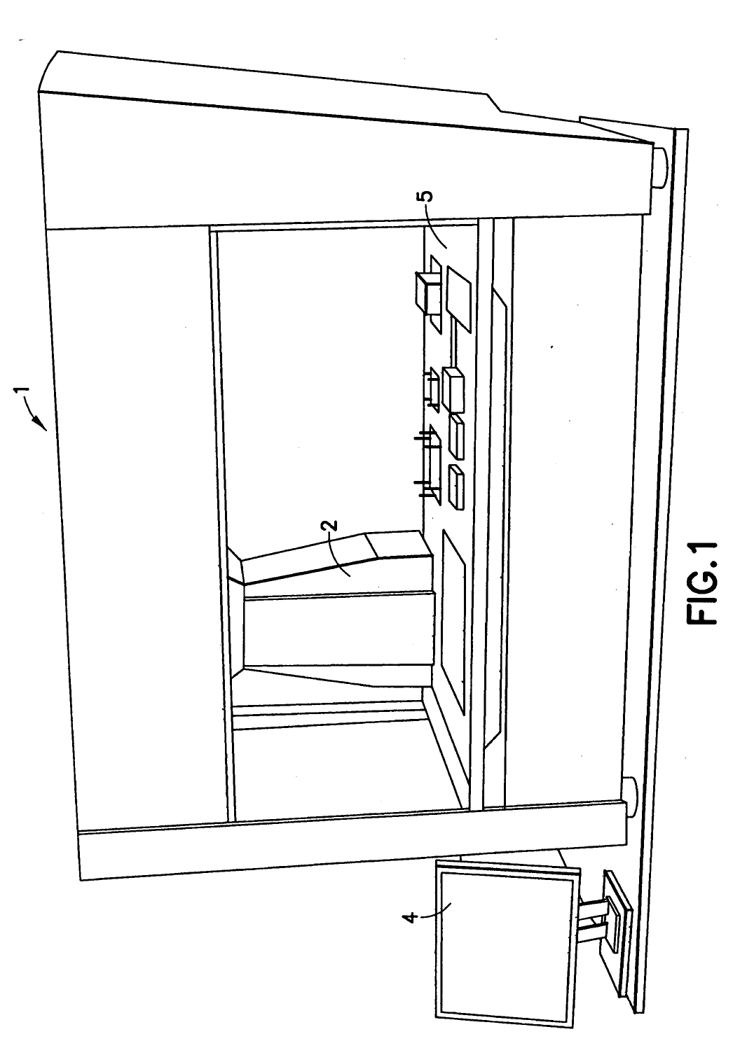

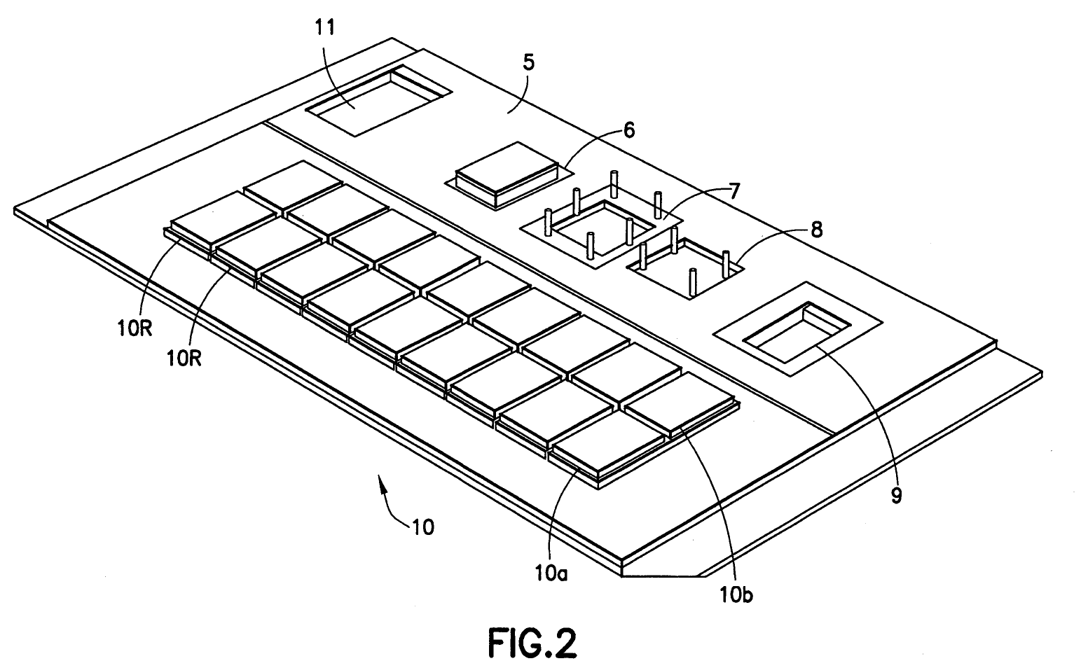

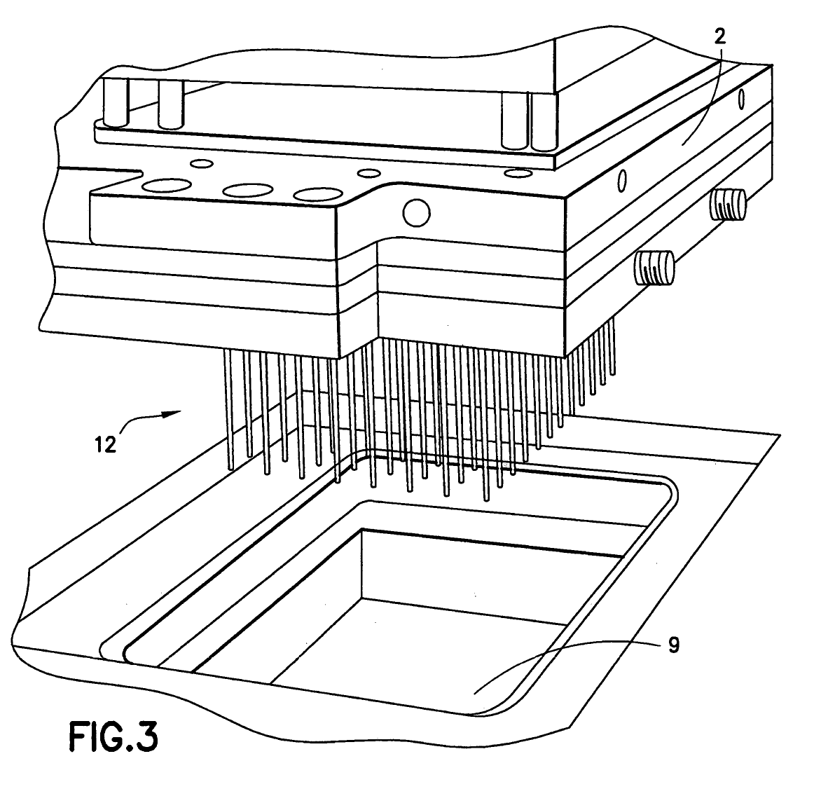

As shown in FIG. 1, the automated processing system 1 employs a robot 2 controlled by a computer 3 having a user interface in the form of a display 4. An example of a system of this type is a processing system sold under the trademark PARALLAB™ by Brooks Automation, Inc. The user interface 4 provides interactive features that allow the operator to select or enter various process parameters as desired. The robot 2 is moveable under control of computer 3 over a work deck 5. Work deck 5, as shown in FIG. 2 has multiple process stations 6-11 positioned on the work deck 5 to allow robot 2 to access each station with in its convenient range of movement. The particular arrangement of FIG. 2, is for illustration and any pattern and combination of processing stations can be used depending on the biotechnical process being performed. The process stations 6 may include any desired number of reagent nests or storage stations 10R, as well as acid bath 10a and base bath 10b stations (see FIG. 3). It is advantageous to position wash station 7 in proximity to the acid bath and base bath stations 10a and 10b to minimize robot motion during the decontamination process. Though only one respective acid and base station is shown in FIG. 2, in alternate embodiments there may be any desired number of different acid and base stations, such as for storing different concentrations of acid and bases.

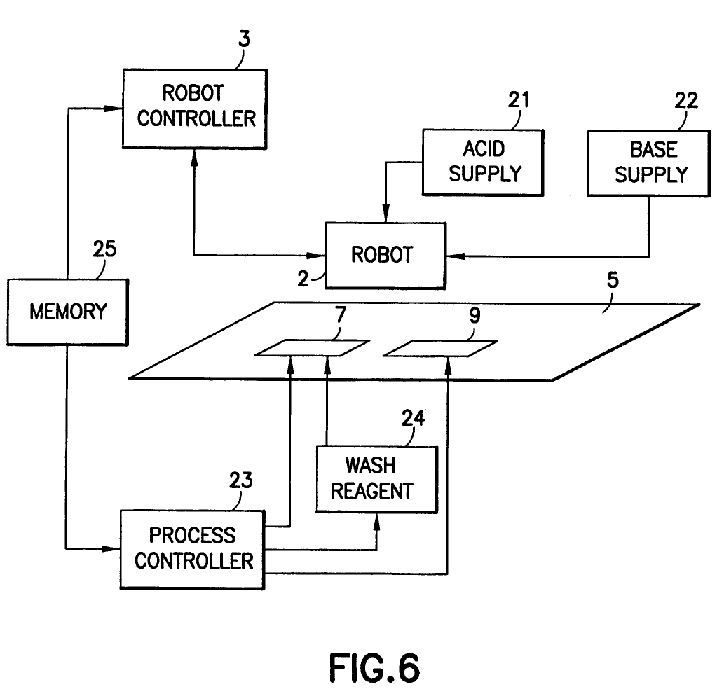

Depending on the application methods used for the acid and base solutions, it may be advantageous to connect the robot to the supplies 21 and 22, as shown in FIG. 6, to allow application of the decontamination solutions to the reaction vessels. This may be the case with larger vessels. In this embodiment, the robot 2 would be moved to the wash station 7 for the application of the contamination solutions. In the embodiment shown in FIGS. 2-3, the reaction vessels consist of an array of capillary tubes 12. Introduction of Decontamination solutions into tubes 12 in this case is by aspiration of the acid and base solutions into the reaction vessels as will be described further below.

As noted before, the process stations include multiple reagent nests 10R for sample and reagent storage. The process stations 10 also may include an acid bath station 10a and a base bath station 10b, as part of a decontamination section having multiple work stations constructed on work deck 5 to provide various functions of the decontamination process described herein. The cleaning process of system 1 is performed by moving the robot among the acid bath station 10a, wash station 7, thermal cycle station 9, and base bath station 10b.

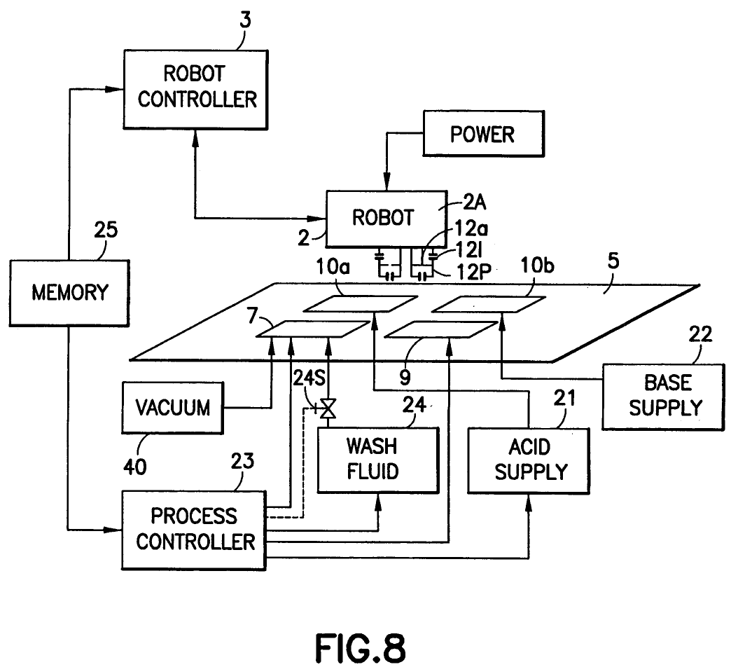

The array of capillary type reaction vessels, such as nano-pipetter 12 are supported for movement with robot 2, as shown in FIG. 3. Robot 2 is mounted on an appropriate drive mechanism (not shown) to provide controlled movement over the surface of the work deck 5. This movement would be lateral in a plane parallel to the plane of the work deck and as well as up and down to allow presentation of the reaction vessel array 12 to the selected process station. The array of vessels in the nano-pipetter 12 may be of any desired size, and the reaction vessels in the nano-pipetter 12 may also be of any desired size. A representative vessel 12P of the nano-pipetter array is schematically illustrated in FIG. 8. In this embodiment, the reaction vessel 12P has an opening for aspiration/discharge of fluids, and a movable plunger 12a located inside the vessel. The plunger 12a is engaged by an actuator 2A in the robot capable of moving the plunger 12a up/down in the reaction vessel. The actuator 2A may be of any suitable type such as an electric motor (e.g. servo motor or stepper motor) or a pneumatic actuator powered from a suitable power supply connected to the robot. Actuation of the plunger generates the desired head in the reaction vessel for aspiration and discharge of fluid into the vessel through the vessel base opening. In alternate embodiments, the robot may have any other desired actuation system for introducing fluid into the reaction vessels, including vessels without a movable plunger located therein. The vessel 12P also includes, as seen in FIG. 8, a flushing port 12I through which fluid may be introduced for flushing the vessel. In FIG. 8, the plunger 12a is in a position closing the flushing port 12I and enabling aspiration/discharge through the base fluid opening. The actuator 2A is capable of raising the plunger to a third position (i.e. a different plunger position than the aspiration and discharge positions of the plunger) uncovering the flushing port 12I and enabling flushing of the reaction vessel.

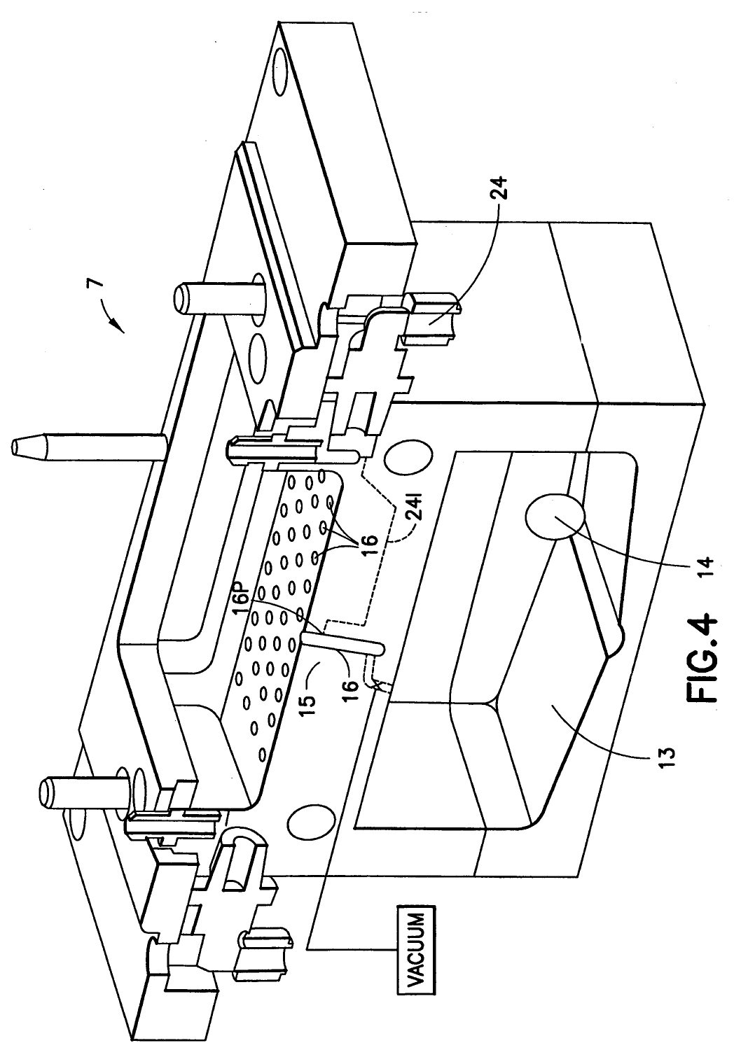

Wash station 7 is shown in FIG. 4 and is constructed having a wash chamber 13 with a waste basin and outlet 14. A supply 24 of wash fluid (such as laboratory quality water) is connected to wash chamber 13, as shown in FIGS. 4 and 8. Block 15 is constructed having multiple apertures or tubes 16 to accommodate the reaction vessels 12 in a supported position during the cleaning operation. Each aperture 16 has a wash feed port 16P, schematically shown in FIG. 4, located to contact and form fluid communication with the reaction vessel flushing port 12I, when the reaction vessel 12, 12P is positioned by the robot into the corresponding aperture 16 of the wash station block 15. The wash feed port 16P, as may be realized is connected via a suitable manifold or wash fluid distribution system (not shown) to the wash fluid 24. The wash fluid system has a suitable control valve 24S (e.g. a solenoid valve) operated by the controller 23 to control flow to the wash feed port 16P. Each aperture 16 may also have a waste port or opening for discharging fluids emptied from the reaction vessels, or otherwise introduced into the aperture 16, into the waste basin. The waste basin may be connected to vacuum source 40 to facilitate emptying of the apertures 16 and waste basin. Robot 2 enters wash station 7 and inserts the reaction vessels 12 into the chamber 13 through apertures 16 to perform the initial steps of the cleaning process. During the decontamination process the robot 2 is programmed to move from wash chamber 7, where process fluids are evaluated, to acid bath station 10a, where the reaction vessels 12 are filled with an acid solution, after which the filled reaction vessels 12 are moved and inserted into thermal cycle station 9, where they are subjected to heat.

Thermal cycle station 9 is constructed having a heating chamber 17 into which hot air is forced. A fan 18 directs air though passage 19 over heating element 20 and into chamber 17 to heat the reaction vessel array 12. In an alternate embodiment, any type of heating system may be used.

As shown in FIG. 6, the motion of robot 2 is controlled by a processor 3 which executes motions as directed by an algorithm resident on memory 25. The motion of the robot 2 is coordinated with process controller 23 which controls the flow of fluids such as wash reagent from supply 24, acid from supply 21, and base from supply 22 according to an algorithm resident on memory 25. The operation of the washing station 7 and thermal station 9 is also controlled by process controller 23 according to the process algorithm.

The robot actuator 2A is controlled to cycle the vessel plunder operation in a manner that provides a movement of the acid or base solution within the capillary tubes of the reaction vessel array 12. This provides aspiration/discharge of the acidal base solution into the vessel, as well as an agitation of the decontamination solution within each reaction vessel to insure full coverage of the surfaces of the reaction vessel subjected to contamination. The cycle of operation for pump 26 would be based on the physical shape, height and volume of the reaction vessel used. A control algorithm is provided to provide the appropriate plunger motion cycle including timing actuator motor commands. Data relating to the height of the capillary tube for which contamination solution coverage is desired may be provided from data derived from the PCR process or from data entered by the user at operator interface 4. The control algorithm may be designed to determine or identify the plunger cycle desired from the data available. The control algorithm may include suitable information (i.e. look up tables or algorithms) to establish the cycle parameters, as well as other possibly variable cleaning operation parameters, from the selection input of the user, such as for example selecting between different types and sizes of reaction vessels. In alternate embodiments, the robot may be equipped with sensors for detecting the type of vessel in the nano-pipetter array and communicating the information to the controller, for use by the control algorithm.

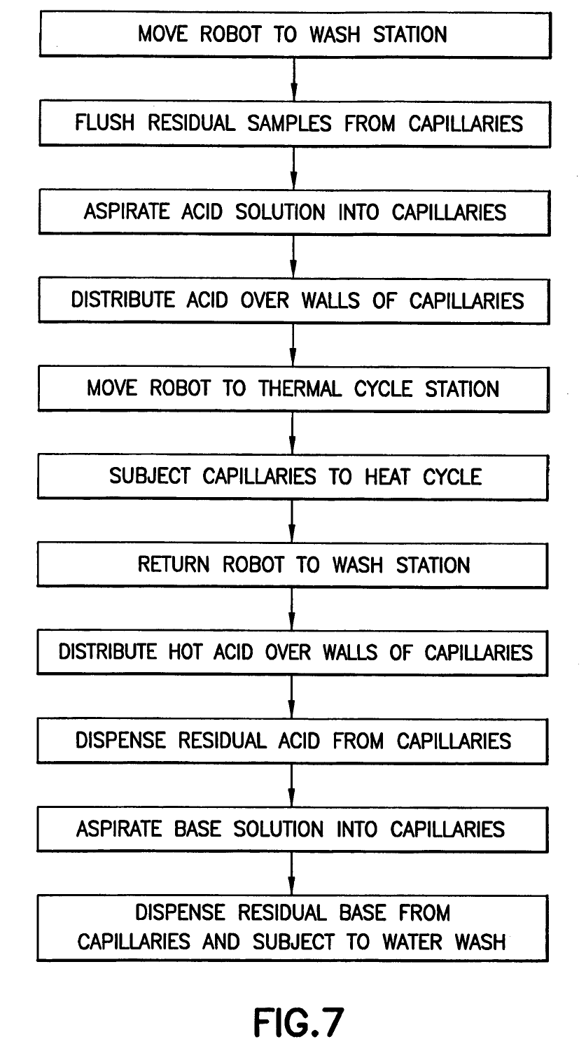

In operation the cleaning cycle may be initiated at any desired time such as after completion of a process cycle, such as the polymerase chain reaction mentioned above. A suitable decontamination protocol, an example of which is shown in Table 1 below and in the steps of FIG. 7, is programmed into controller 23. In accordance with the programming, the robot, holding the process contaminated reaction vessel array 12, is moved to the washing station 7 and residual samples are dispensed from the capillaries by plunger action possibly in combination with vacuum suction in tubes 16. A solution of nitric acid is then dispersed into the capillaries in a manner that covers the walls of the capillaries.

In one embodiment, as shown in FIG. 8, this is accomplished by moving the robot 2 with the reaction vessels 12 to an acid bath station 10a constructed in the work deck 5 as one of the multiple process stations. The acid solution is supplied to the acid bath station 10a and is aspirated into the reaction vessels by the action of plunger 12a. It may be advantageous to include an agitation step by cycling the plunger 12a up and down with the vessel 12 for the desired number of cycles in order to insure full distribution of the acid. Such an agitation step may be accomplished at wash station 7 to avoid spillage and possible contamination of the acid supply. In an alternative embodiment the agitation step could be accomplished by application of ultrasound vibrations to the reaction vessel array 12.

As previously described, in the embodiment shown in FIG. 6, the acid solution is supplied through the robot 2 while the reaction vessel array 12 is positioned in the wash station 7.

The acid filled reaction vessels 12 are moved by the robot to the thermal station 9 where heat is applied, according to the specifications suggested in Table 1. After the heat cycle is complete, the robot moves the reaction vessels 12 back to the wash station where further dispersion of the hot acid may be accomplished by agitation or other means. The acid is then dispensed from the reaction vessels 12.

The next step in the process is to disperse a base solution of Potassium Hydroxide into the reaction vessels 12 taking care to distribute the base solution fully along the walls of the reaction vessels 12. This is accomplished by causing the robot to move from the wash station to a base bath station 10b. Bath station 10b is also constructed on work deck 5 as a further process station. The base solution is then aspirated by operation of the plunger 12a in the reaction vessels 12. The base solution is then moved back to the wash station where it is dispensed from the capillaries and flushed. An agitation step may also be included, prior to flushing, by cycling the plunger 12a for the desired cycles to insure sufficient coverage of the contaminated reaction vessel. Again this would be accomplished at wash station 7.

In the application of either the acid or base solutions, a predetermined amount of such solutions may be obtained within the reaction vessels by appropriate timing of the operation of the vacuum pump. This would be governed by the pump control algorithm based one reaction vessels parameters entered or otherwise available.

In an alternate embodiment a strippable coating is applied to the capillaries prior to sample processing. A silinization coating may be used for this purpose The strippable coating is designed to be removed during the decontamination process.

TABLE 1

| Process Step Description | Quantity |
|---|---|
| Dispense any remaining sample in the | |
| capillaries | |
| Aspirate nitric acid | 4000 nl of 2 M |
| Pipette the acid up and down the length | 10 cycles |
| of the capillary walls which were | |
| exposed to the sample | |
| Heat in thermal cycler | 3 mins at 95 C. |
| Pipette the hot acid up and down the | 10 cycles |
| length of the capillary walls which | |
| were exposed to the sample | |
| Dispense out the contents of all the | |
| capillaries to waste | |
| Aspirate potassium hydroxide | 6000 nl of 0.2 N |
| Pipette up and down the length of the | 10 cycles |
| capillary walls 10 cycles which were | |
| exposed to the sample | |
| Dispense out the contents of all the | |
| capillaries to waste | |
| Dock on wash station, extract plungers | 3 minutes |
| and flow DI water wash through pipetter | |

It should be understood that although the above protocol for a particular embodiment is described with specific concentrations and duration times, a wide variety of parameters may be used and are intended to be within the scope of the invention.

To demonstrate the efficacy of the cleaning protocol, a testing protocol was developed to both ensure that there is no carryover and that the reagents used for decontamination would not inhibit the next reaction.

### EXAMPLE 1

In a PCR decontamination experiment, all the channels of a nano-pipetter were filled with DNA template and PCR cocktail (primers, nucleotides, polymerase and buffer). The sample was subjected to thirty-five complete thermal cycles (“+” reaction). The sample was removed from the vessel and run on a gel. Upon completion of the PCR reaction, the nano-pipetter was cleaned using the cleaning protocol of Table 1.

After completion of the cleaning protocol, PCR cocktail without template was aspirated into the cleaned vessels and the sample was subjected to thirty-five complete thermal cycles (“−” reaction).

The sample was removed from the vessel and run on a gel. The nano-pipetter was again cleaned using the cleaning protocol of Table 1.

The rounds of PCR amplification were repeated 10 times in the order of +, −, +, − for a total of 20 PCR reactions total. The final PCR + and − reactions in the 10th round were “over-cycled;” i.e., cycled through 45 thermal cycles, instead of 35.

Table 2 shows the results of rounds 1, 4, 7, and 10 as well as too 100 bp ladders. The data shows that, in the absence of added templated, there was no amplified material appearing on the gel, thus demonstrating that there was no template carried over from the preceding round containing template.

The results demonstrate that there is no measurable trace of DNA template or amplicons left over from precious PCR reactions agter the cleaning protocol is performed (no product in the “−” lanes); the cleaning protocol does not inhibit subsequent PCR reactions (strong signal in all the “+” lanes); and there is no cumulative effect after 20 cleanings.

[image]

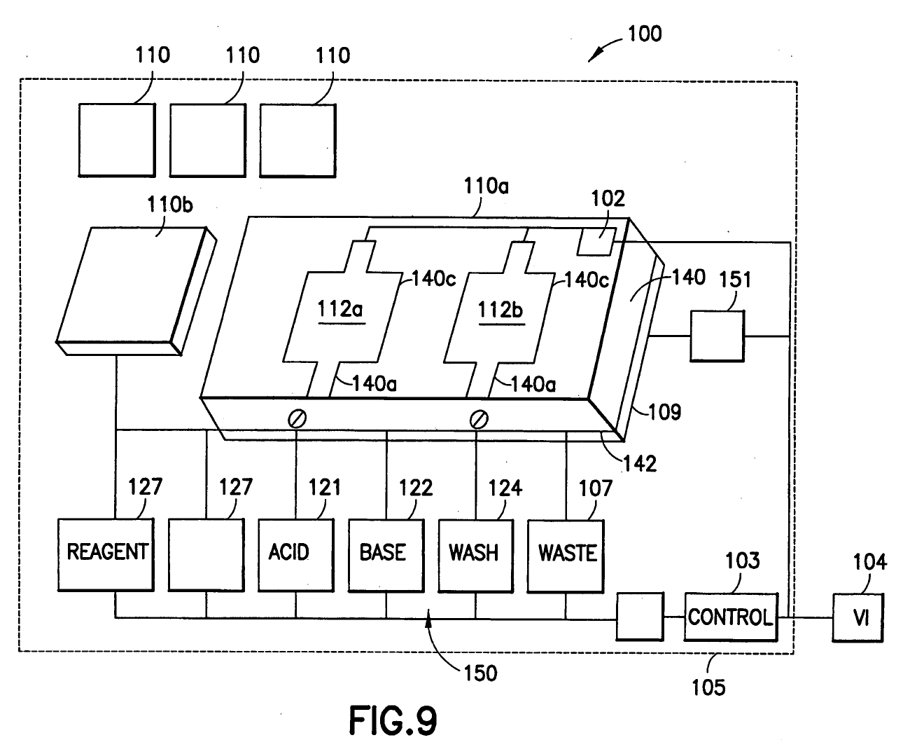

Referring now to FIG. 9, there is shown a schematic illustration of automated system 100 in accordance with another exemplary embodiment. Except as otherwise noted, system 100 generally similar to system 1 described before, and shown in FIGS. 1-8. Similar features are similarly numbered. As seen in FIG. 9, system 100 is has a frame 105 with one or more processing stations 110, 110a, 110b located thereon. The processing stations may be substantially similar to each other, or may be different as desired. Reaction vessels 112a, 112b may be located at one or more of the processing stations. The reaction vessels 112a, 112b may be connected to reagent solution supply sections on the frame for carrying out desired biotechnical processes such as PCR. Similar to system 1, system 100 also includes a controller 103, with a suitable user interface 104. The controller, which may be connected to frame 105, is suitably interfaced with the reaction vessels 112a, 112b and the reagent supplies to automatically carryout the biotechnical process in vessels 112a, 112b. Also similar to system 1, system 110 has an integral reaction vessel decontamination system 150 operated by the controller 103 for the in situ decontamination of the reaction vessels 112a, 112b to a degree sufficient to allow reuse in biotech processes such as PCR.

In the embodiment shown in FIG. 9, the reaction vessels 112a, 112b are contained in a micro-fluidic chip type apparatus 140. In this embodiment the processing stations 110a, 110b are capable of holding one or more micro-fluidic chip apparatus 140 (only one apparatus 140 is shown for example purposes). The micro-chip apparatus 140 may have suitable. micro-chambers 140c and vias 140a defining the reaction vessels 112a, 112b and feed/discharge channels for the reaction vessels. When located in the processing station, the vias of the micro-fluidic chip 140 may be connected to complementing feed/discharge ports (not shown) of a fluid channelizing system 142 of the system 100, which in turn connects the reagent supply 127, and the waste station 107 to the reaction vessels 112a, 112b in the micro-fluidic chip. Accordingly, in this embodiment reagent solution from the reagent supply 127 may be introduced via the fluid channelizing system 142 and vias 140a to the reaction vessels 112a, 112b, and process residue or any other fluid may be discharged from the vessels of chip 140 to the system waste 107. As seen in FIG. 9 an actuation system 102 is included to generate the impetus or head for transporting the reagent solution from the supply 127 into the reaction vessels, and for discharging residue from the vessels to the system waste 107. The actuation system 102, which in the embodiment shown, may be located on the chip 140, or in alternate embodiments in any other desired location on frame 105, may be of any suitable type such as pump or vacuum source. In other alternate embodiments the actuation system may provide positive head on the reagent supply to cause flow in to the reaction vessels. In still other alternate embodiments, the actuation system 102 may include or conversely be incorporated into a robot similar to robot 2 in FIGS. 6 and 8 capable of moving the chip apparatus to a dedicated processing station of the apparatus for introduction of the reagent solution in the reaction vessels. As seen in FIG. 9, the actuation system 102 is communicably connected to the controller 103. The controller 103 has programming for open of closed loop control of the actuation system 102.

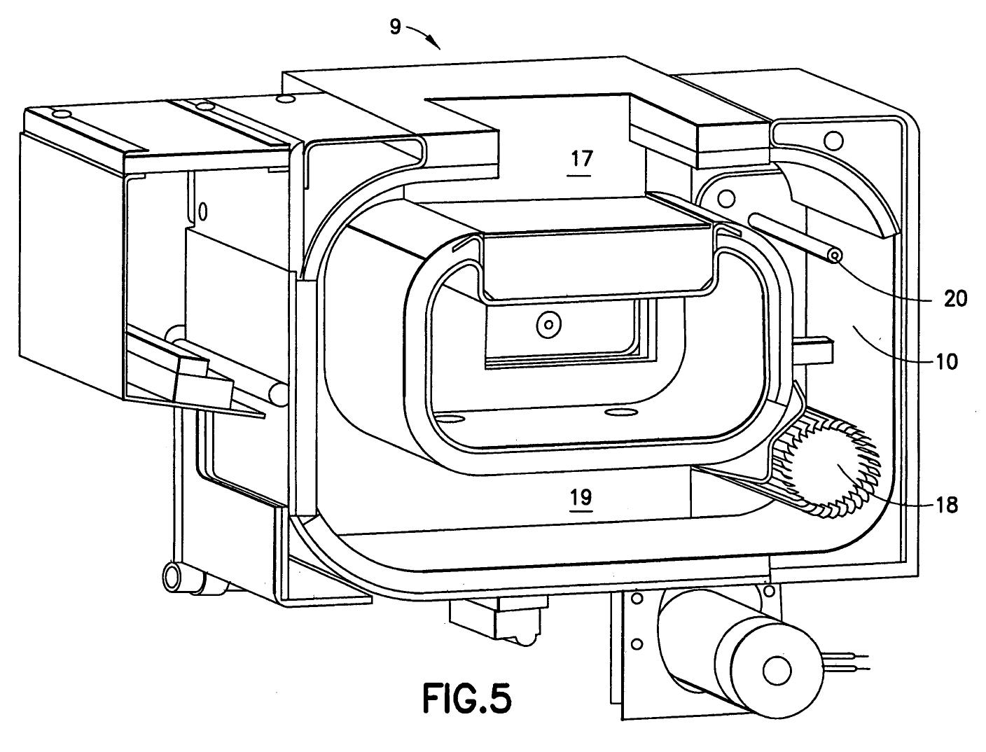

As noted before, system 100 has an integral reaction vessel decontamination system 150 mounted to frame 105. In this embodiment, the system 100 has an acid supply 121, a base supply 122, and a supply of wash fluid 124 located on the frame 105. The system 100 in this embodiment also has a heater 109 and is capable of agitating the fluid in the reaction vessels as will be described further below. A suitable decontamination protocol, for example similar to the protocol listed in table 1 above, is programmed in the controller 103. The supplies of acid 121, base 122 and wash fluid 124 may be located as shown to communicate with the reaction vessels via the distribution system 142. Suitable valving (not shown), controlled by controller 103 may be used to isolate the acid, base and wash fluid supplies from other reagent supplies and the reaction vessels 112a, 112b. To introduce acid, base or wash fluid from the respective supplies, according to the decontamination protocol, the controller 103 commands actuation system 102, and enables access to the desired supply 121, 122, 124, to transfer a program defined quantity of the acid base or wash fluid into the reaction vessels 112a, 112b. Agitation of the acid or base in the reaction vessels similar to the protocol described before may be accomplished by the actuation system 102, under command from controller 103, cycling the acid or base fluid back and forth in the reaction vessels. As seen in FIG. 9, system 100 may also include an agitator 151, such as a mechanical agitator, or acoustic agitator, connected to the chip apparatus 140 to agitate the reaction vessels or fluid therein as commanded by controller 103. The agitator 151, may be integrally formed in the chip apparatus 140. Heating of the acid in the reaction vessels, similar to the earlier described decontamination protocol, is performed by heater 109, as commanded with controller 103. The heater 109 may be integral with the chip apparatus 140. In alternate embodiments, the heater 109 may be mounted to the frame 105, such as for example in a contact or seating surface of the processing section 110a forming good thermal contact with the chip apparatus 140 surface when the chip apparatus is seated in the processing station. In other alternate embodiments, the heater may be located at a different processing station, similar to heating section 9 in FIGS. 2 and 5. In that case, the acid filled chip apparatus may be moved from processing station 110a to the heating section for heating the acid in the reaction vessels.

It should be understood that the foregoing description is only illustrative of the invention. Various alternatives and modifications can be devised by those skilled in the art without departing from the invention. Accordingly, the present invention is intended to embrace all such alternatives, modifications and variances which fall within the scope of the appended claims.

1. A method of reusing reaction vessels in a biotechnical application comprising
providing a biotechnical processing apparatus with at least one processing station for performing a biotechnical process., and an automatic actuator for charging reagents into a reaction vessel to perform a biotechnical process in the reaction vessel at the at least one processing station, the apparatus having an integral cleaning system for cleaning the reaction vessels when contaminated;
with the actuator, introducing an acid solution from the integral cleaning system into the reaction vessel;
with the integral cleaning system, heating the acid solution;
with the actuator, eliminating the acid solution from the reaction vessel;
with the actuator, introducing a base solution from the integral cleaning system into the reaction vessel;
with the actuator, eliminating the base solution from the reaction vessel; and
with the integral cleaning system, flushing the reaction vessel with a wash fluid.

2. The method of reusing reaction vessels, according to claim 1, further comprising agitating the acid filled reaction vessel with the integral cleaning system to cover a predetermined contaminated portion of the reaction vessels with the acid solution.

3. The method of reusing reaction vessels, according to claim 1, wherein introduction of the acid solution comprises operating a plunger with the actuator to aspirate the acid.

4. The method of reusing reaction vessels, according to claim 3, wherein agitation is accomplished by cycling the plunger.

5. The method of reusing reaction vessels according to claim 1, further comprising providing the actuator with a robot for moving the reaction vessel relative to the at least one processing station, moving, with the robot, the reaction vessel to a wash station of the apparatus and dispensing process residues from the reaction vessel;
causing the robot to move the contaminated reaction vessel to an acid station of the cleaning system for aspirating the acid solution into the reaction vessels;
causing the robot to move the acid filled reaction vessels to a thermal station of the cleaning system for heating the acid solution in the reaction vessel;
causing the robot to move the heated reaction vessel to wash station of the apparatus, for dispensing the acid solution from the reaction vessels;
causing the robot to move the reaction vessel to a base station of the cleaning system for aspirating the base solution into the reaction vessel; and
causing the robot to move the reaction vessel to the wash station for dispensing the base solution from the reaction vessel.

6. The method of reusing reaction vessels, according, to claim 2, wherein the reaction vessel containing the base solution is agitated to cover the predetermined contaminated portion of the reaction vessel with the base solution.

7. The method of reusing reaction vessels, according to claim 6, wherein the agitation step is accomplished by cycling a plunger with the actuator.

8. The method of reusing reaction vessels, according to claim 1, wherein the acid is heated to a temperature in the range of 28 to 200 degrees Celsius.

9. The method of reusing reaction vessels, according to claim 1, wherein the acid is nitric acid in a 0.001 to 15 molar solution.

10. The method of reusing reaction vessels according to claim 1, wherein the base is Potassium Hydroxide, in a 0.001 and 5 normal solution.

11. The method of reusing reaction vessels, according to claim 1, wherein the reaction vessel is made of glass.

12. The method of reusing reaction vessels, according to claim 1, wherein the reaction vessel interior is coated with a strippable coating before use in the biotechnical process and wherein the strippable coating is removed during decontamination.

13. The method of reusing reaction vessels, according to claim 12, wherein the strippable coating is a silinization coating.

14. The method of reusing reaction vessels, according to claim 1, wherein the biotechnical process is a polymerase chain reaction.

15. A system for performing biotechnical processing, comprising:
a frame having at least one processing station constructed therein;
an array of reaction vessels mounted to the frame for effecting biotechnical processing at the at least one processing station;
an automatic actuator connected to the frame and said reaction vessels for dispersion of solution into and out of the reaction vessels;
a decontamination system constructed in the frame for being accessed by the actuator to decontaminate the reaction vessels, the decontamination section further comprising:
a wash section connected to the frame to receive and remove fluids from the reaction vessels;
a heating section connected to the frame to heat the reaction vessels;
a supply of wash fluid, a supply of acid solution and a supply of base solution connected to the frame for application to the reaction vessels; and
wherein said actuator is programmed to access said decontamination section to subject the reaction vessels to: an acid solution, heat, a base solution, and a wash reagent for decontaminating the reaction vessels.

16. The system for performing biotechnical processing, according to claim 15, wherein the decontamination section further comprises:
an acid bath station constructed in the frame and connected to the supply of acid solution; and
a base bath station constructed in the frame and connected to the supply of base solution.

17. The system for performing biotechnical processing, according to claim 15, wherein the wash station comprises:
a retaining block having apertures constructed therein, the apertures being formed to accommodate the passage of the reaction vessels; and
a wash chamber accessible to the reaction vessels through the apertures and connected to flush the acid solution, base solution, and wash fluid applied to the reaction vessels.

18. The system for performing biotechnical processing, according to claim 15, wherein the heating station comprises.
a heating chamber to accommodate the array of reaction vessels; and
a source of heated air connected to circulate heated air into the heating chamber.

19. The system for performing biotechnical processing, according to claim 18, further comprising a process controller with programming arranged so that upon execution, the programming causes said system to perform a decontamination process comprising:
causing the actuator to dispense residue from said array of reaction vessels into the wash section;
causing the actuator to access supply of acid solution to fill said reaction vessels with the acid solution;
causing the actuator to interface with said heating section to heat the acid solution filled reaction vessels;
causing the actuator to flush the acid solution from the reaction vessels into the wash section;
causing the actuator to access the supply of base solution to fill the reaction vessels with base solution;
causing the actuator to flush the base solution from the reaction vessels into the wash section; and
causing the actuator to access the supply of wash fluid to flush the reaction vessels.

20. The system for performing biotechnical processing, according to claim 19, wherein the heated acid solution in the reaction vessels is agitated by at least one of the actuator or the decontamination system to cover a contaminated portion of the reaction vessel with the acid solution.

21. The system for performing biotechnical processing, according to claim 19, wherein the base solution in the reaction vessels is agitated by at least one of the actuator or the decontamination system to cover a contaminated portion of the reaction vessel with the base solution.

22. The system for performing biotechnical processing, according to claim 19, wherein the acid is heated in the heat station to a temperature in the range of 28 to 200 degrees Celsius.

23. The system for performing biotechnical processing, according to claim 19, wherein the acid is nitric acid in a 0.001 to 15 molar solution.

24. The system for performing biotechnical processing, according to claim 19, wherein the base is Potassium Hydroxide, in a 0.001 and 5 normal solution.

25. The system for performing biotechnical processing, according to claim 19, wherein, the reaction vessels are made of glass.

26. The system for performing biotechnical processing, according to claim 19, wherein an interior of the reaction vessels is coated with a strippable coating and wherein the strippable coating is removed during the method of decontamination.

27. The system for performing biotechnical processing, according to claim 26, wherein the strippable coating is a silinization coating.

28. The system for performing biotechnical processing, according to claim 19, wherein the actuator comprises a robot movably connected to the frame and capable of holding the reaction vessels to transport the vessels between at least the wash section and heating section, wherein the robot is capable of applying a vacuum to the array of reaction vessels too aspirate the acid solution and the base solution into the reaction vessels.

29. The system for performing biotechnical processing, according to claim 15, wherein an agitator is provided to agitate the reaction vessels after they are filled with the acid solution and again after they are filled with the base solution.

30. The system for performing biotechnical processing according to claim 15, further comprising a controller communicably connected to the actuator and decontamination system, wherein the controller comprises a control algorithm for repetitive cycling of at least one of the actuator or the decontamination system to insure coverage of the reaction vessels by the acid solution and the base solution.

31. The system for performing biotechnical processing, according to claim 15, wherein the biotechnical processing comprises a polymerase chain reaction.

32. The method of decontaminating reaction vessels, according to claim 4, wherein the agitation means comprises a source of ultrasound vibrations for application to the reaction vessels to insure coverage of the reaction vessels by the acid and base solutions.
